(19) United States
(12) Patent Application Publication
(10) Pub. No.: US 2018/0185194 A1
(43) Pub. Date: Jul. 5, 2018

(54) Rejuvenation of Billions of Eyelids and Eyes

(71) Applicant: Anuthep Benja-Athon, New York, NY (US)

(72) Inventor: Anuthep Benja-Athon, New York, NY (US)

(21) Appl. No.: 15/396,788

(22) Filed: Jan. 2, 2017

(51) Int. Cl. A61F 9/007; A61F 9/00

(52) U.S. Cl. CPC A61F 9/007; G06F 19/324; A61F 9/0008

(57) ABSTRACT

A system and method for preventing and reducing droopy and puffy eyelids and for preserving and restoring visual acuities and preventing and reducing presbyopia and visual losses. The system comprises an apparatus having diverse structures for modulating and controlling the circulations and volumes of blood and fluids in eyelids, eyes and surrounding soft tissues and a means for avoiding the compression and disruptions of the circulations and physiology of the corneas and lenses, networked repositories for predicting and extrapolating trends of developments of said conditions and the needs of utilizations of said system and networked machines for producing individual customized apparatuses for fitting said organs and tissues of individuals.

### FIELD OF THE INVENTION

A system and method for rejuvenating eyelids and eyes

### BACKGROUND OF THE INVENTION

In reconstruction surgery and the likes, the techniques of iatrogenic stretching of skin in various parts of the body are well known and described. The surgical techniques involve the use of various types of skin and tissues expanders to cause stretched skin.

In nature, similar phenomenon occurred in billions of people. Some common examples are sagging eyelids, distended abdominal skin in pregnancy, beer belly and obesity. Shrinkage of said skin do occur. In aging, eyelids stretch and the muscles supporting them weaken and excess adipose tissue in the upper and lower eyelids, causing sagging eyebrows, droopy upper lids and lower lids aka eyebags.

The present invention identified hemodynamics of the arterial, venous and lymphatic circulations, body fluids and tissues such as, but not limited to, blood components and cells—particularly when and during the time people are recumbent and sleeping—being brought in by the blood vessels into and around said organs and tissues and resultant physiology involving the proliferation, growth, expansion and changes of soft tissues such as, but not limited to, muscles, connective tissues and adipose tissues in and posterior to the eyelids to cause said conditions described herein.

For example, first, increase quantity of fluid, connective tissues and adipose tissues in the upper and lower eyelids cause age-related stretched, droopy and sagging upper and lower eyelids commonly seen in middle-age and older people—see television and photographs—the results of stretched, excess, sagging and physiological changes of the skin, muscles, soft tissues including connective tissues and adipose tissue and fluid in said organs and tissues. Sagging eyelids can reduce peripheral visions in particular upper and outer fields of vision. Although blepharoplasty can reduce or eliminate these vision problems and make eyes appear younger and more alert, it is expensive and not without risks, complications, morbidity and mortality. Recurrences of said conditions are common.

Second, visual changes such as, but not limited to, age-related presbyopia aka farsightedness and the likes—inevitably, in middle-age and older people—appeared to be age-related. More than 150 million Americans of age 40 and older (US Census Bureau, 2014). A hypothesis—presbyopia involved the changes of the lens proteins, making the lens harder and less elastic over time and the intra-ocular muscle fibers surrounding the lens. Other hypotheses exist. Yet the present hypothesis is based on the observation that presbyopia may share the same hemodynamics and physiology causing said age-related eyelids.

Billions of people, inevitably, have said common conditions. They appearing and feeling older and less functional and more prone to loss of productivity, accidents and injuries due to visual changes and handicaps being imposed by the necessities to use eyewears, contact lenses and surgeries and intangible losses such as, but not limited to, pain and suffering. The socioeconomic and psychological implications and consequences causing $billions of losses per annum. Not to mention the immense intangible losses. Furthermore sagging skin around your eyes can reduce visions such as, but not limited to, peripheral visions.

The present invention, first, retards and prevents said conditions in any and all idiosyncratic, inborn, different and diverse head and facial anatomy of all humans;

Second, indicates the optimal management such as, but not limited to, providing analgesics and anti-inflammatory modalities to reduce pain and significantly reduced inflammation and to support the integrity of the surgical incisions and sites to prevent inadvertent disruptions and post-surgical complications of surgical sites and the surrounding soft tissues and to promote rapid healing of the surgical sites and the surrounding soft tissues in patients as surgical dressing at the time after the operations in the operating rooms to home in the days and weeks after the blepharoplasty; third, promotes, accelerates and attains skin shrinkage in any conditions; and fourth, prevents and retards recurrences of said conditions after said surgeries in individuals.

The tangible and intangible benefits for billions of people are immense and forever.

### SUMMARY OF THE INVENTION

Eyebags and presbyopia heretofore eternally inflicting socioeconomic losses, pain and suffering and disrupting and limiting activities of living, visions, function and longevity of billions of people. A system and method for preventing and reducing droopy and puffy eyelids related to aging, in post-blepharoplasty and post-surgeries of eyelids and for preserving and restoring visual acuities and preventing and reducing presbyopia and visual losses. The system comprises an apparatus having several embodiments for modulating and controlling circulations and volumes of blood and fluids in the eyelids, eyes and soft tissues. The system further comprises networked repositories for rendering data and analyzing said data for predicting and indicating trends and developments of said conditions and the needs of utilizations of the system by people. Furthermore, said networked repositories for instructing networked system of ubiquitous 3-D printers for rendering individual customized apparatus for fitting individual idiosyncratic dimensions, sizes and anatomical contours and relative positions of said organs and tissues of individuals based on specifications and instructions rendered from analytics of said data by said networked repositories.

### DESCRIPTION OF THE PREFERRED EMBODIMENTS

The unique discovery of the present invention is the results of clinical research and basic medical science research performed by and known only to this physician applicant. The basic medical sciences are buried in the Textbook of Anatomy. W. H. Hollinshead 2nd ed., Chapter 27, 1967 wherein the vascular of the eyes and eyelids are described in detail. The basic sciences on the hemodynamics and physiology are buried in the Medical Physiology, V. B. Mountcastle 13th ed., Cellular Physiology Part I and Circulation Part Vi-Part Vii 1974.

The present invention is a system and method for preventing and reducing sagging, droopy and puffy eyelids, pre- and post-blepharoplasty and pre- and post-surgeries of eyelids and visual changes and losses. The present invention is based on said hemodynamics and physiology of and around the eyes, in particular, but not limited to, the cornea, lens, aqueous humor, irido-corneal angle, ciliary body and process, suspensory ligament and vitreous humor henceforth collectively known as eyes, extra-ocular muscles, orbicularis oculi and surrounding soft tissues such as, but not limited to, adipose tissue, connective tissue and elastic tissue in and around said organs and surrounding soft tissues henceforth collectively known as soft tissues based on said clinical and basic sciences.

Said system comprising an apparatus for modulating and controlling the circulations and volumes of blood and fluids in said eyes, eyelids and related soft tissues and said means attaching to a means for implementing said means. Said apparatus—described the claims of the invention herein—comprising, but not limited to, several preferred embodiments, structures, configurations and devices for fitting to idiosyncratic dimensions, sizes and peculiar anatomical contours and positions of said organs, bony and soft tissues of people in diverse and different settings and circumstances on earth.

Said apparatus for preventing and reducing the accumulations of fluids and undesirable growth, proliferations and expansion of said organs and soft tissues in or around said organs and soft tissues. In other words, the system for preventing and reducing droopy and puffy eyelids aka eyebags, pre- and post-blepharoplasty and pre- and post-surgeries of eyes and eyelids. The system also promotes, accelerates and attains skin shrinkage in any disease and normal conditions. Additionally, the system also prevents and reduces losses of visions being caused by said sagging, droopy and puffy eyelids and also prevents and reduces losses of visual acuities such as, but not limited to, presbyopia based on said physiology and hemodynamics.

The omnipotent system comprises omnipresent networked servers, smartphones, laptops and desktops having first networked repositories comprising networked processor units attached computer programs instructing said first networked repositories how to receive and analyze data on demographics, genetics, phenotypes, lineages, facial and head images including selfies and videos including selfies in natural and disease-related conditions submitted by said networked communication devices by people on earth. Said first networked repositories having computer programs for predicting and extrapolating trends of developments said conditions in people and their parents, siblings and genotypically related people sharing same genetic and phenotypic lineages anywhere on earth. Consequently, first networked repositories to indicate the needs, circumstances and time of utilizations of said apparatus in individuals. Said data also include, but not limited to, prescription data on said analgesics, anti-inflammatory drugs, antibiotics and any combination thereof related to patient users by physicians.

A case in point, naturally by evolution design, male and female members in individual families most often look alike and, in other words, have same or similar phenotypic and anatomical contours, sizes, shapes and positions of their heads, faces, eyes, eyelids, noses and ears, etc. and likewise the individual peculiarities of said hemodynamics and physiology. Said example, naturally by evolution design, is applicable to individual ethnicities on earth. For example, sagging, droopy and puffy eyelids occurred among parents and siblings within individual families and relatives.

So, with said data being rendered by said networked repositories, said computer programs instructing said first networked repositories what to and how to predict and extrapolate trends of occurrences and prevalence of said conditions in any individual families and relatives individual ethnicities and people and their parents, siblings, relatives and genotypically related people sharing same genetic and phenotypic lineages and of various ages and stages of life and diseases and disorders anywhere on earth. In other words, said first networked repositories can reliably predicting and extrapolating said trends of said conditions in billions of people and to get said susceptible people to start using said apparatus at either the beginning of said conditions to prevent the progress of said conditions and/or mid to late decades of life when said conditions exist to prevent the progress or even reverse said conditions as described herein. Moreover, the apparatus is a must-use for any and all patients who had post-blepharoplasty and post-surgeries—including procedures not mention herein—of eyes and eyelids. One of the facts, as a versatile and dynamic organ, skin will stretch and remain sagged, droopy and puffy when repeatedly and chronically stressed and stretched in said settings and circumstances. Thus, the blepharoplasty and the likes are not all for naught thanks to the present invention. With the application of said apparatus, said stretched skin—a versatile and dynamic organ—can be shrunken thus rejuvenation by the application of said apparatus. Yet another benefits is to prevent and retard recurrences of said conditions after said surgeries.

Said system provides an apparatus comprising a plurality of different structures for serving the same function for achieving the goals and objectives described herein. The detailed descriptions of the preferred embodiments of said structures are well described in the claims of the invention. Therefore, further to said descriptions, the means for modulating and controlling comprises pads, patches and inflatables configured to approximate different surface areas—2 dimensions—of upper eyelid and lower eyelid with a range of the widths—fully open eye to fully closed eye—of the palpebral fissure. Said pads, patches and inflatables are configured to account for the cornea-area-configured spaces between said upper pads, patches and inflatables and lower pads, patches and inflatables in said applications.

However, With said networked repositories networking to or networking to networked repositories for instructing 3-D printers for rendering individual customized pads, patches and inflatables to precisely or best fitting individual idiosyncratic dimensions, sizes and contours and anatomical positions of individual upper eyelids and lower eyelids and surrounding bony and soft tissues of individual users of said networked repositories based on instructions and specifications derived from analyses of said data rendered in and by said networked repositories. Furthermore, said cornea-area-configured spaces between said upper pads, patches and inflatables and lower pads, patches and inflatables are precisely or best rendered by said 3-D printers renderings.

The thickness—the 3rd dimension—of said upper and lower pads, patches and inflatables is no more than ½ inch or 1 inch. Said range of thicknesses is a function or a plurality of functions being served by said upper and lower pads, patches and inflatables. For example, first, pads, patches and inflatables are thin albeit strong and flexible to serve the functions described herein. Second, however, said pads, patches and inflatables will be thick if, for example, said inflatables which are, but not limited to, synthetic bladders being filled with anti-modalities such as, but not limited to, water, gel, chemical beads and air which can be microwaved and refrigerated. Another preferred embodiment—said pads and patches can be impregnated with medications such as, but not limited to, topical analgesics, anti-inflammatory drugs, antibiotics and any combination thereof.

Best with said networked system of 3-D, however, commercial machines—with old and new technologies—in factories and machines in people's homes can also manufacture said pads, patches and inflatables as spares and for attach and be incorporated into ordinary headgears and eyewears—sunglasses, prescription glasses and swim goggles, etc.—owned by people. Various types of attachment means such as, but not limited to, many commercial glues, adhesives, Velcro, etc.

Various types of man-made materials such as, but not limited to, plastic, metals, alloys and glass and any combination thereof are used by 3-D printers and other machines to manufacture said apparatus.

The system also provides an app for detecting and measuring compressive forces being exerted by individual apparatuses on individual users' eyes, eyelids, noses and ears, etc. From said researches, this patent applicant discovered the correct and optimal compressive forces to be rendered by said apparatus in the conditions, settings and circumstances described herein and how to avoid undesirable compression and disruptions of the physiology such as, but not limited to, the stresses and oxygenation of said eyes.

The system, first, provides mobile devices app to adapt said touch-sensitive thus pressure-sensitive screens to detect and measure compressive forces being rendered to said organs and tissues by said structures upon said screens contacting said structures and to display measurements as digital data and voice data. Said screens are made to come into contact with said individual apparatuses to get the digital data being generated and displayed as digital data and voice data by said mobile devices.

Second, said mobile devices comprising electromagnetic wave-measuring devices, said devices to detect and measure the intensity of electromagnetic waves—acceptable level and more or less intensity—being emitted by said organs and tissues being compressed by said structures and to display measurements of said emissions as digital data and voice data. Said first networked repositories managing and disseminating said rendered data in said networks.

Of course, it is obvious to those skilled in the art and science that various other technologies and devices for detecting and measuring force will be applied to the present invention.

The present invention provides second networked repositories networking to said first networked repositories. Said second networked repositories are attached to and instructing said ubiquitous 3-D printers for rendering individual customized said apparatus for fitting individual faces, eyes and eyelids, said second networked repositories comprising computer programs for instructing said 3-D printers to render said individual customized means for modulating and controlling and means for implementing for fitting individual idiosyncratic dimensions, sizes and peculiar anatomical contours and positions of said organs and tissues of individuals based on instructions and specifications rendered by said first networked repositories

Said second networked repositories instructing said 3-D printers to render relative positions of said individual customized means for modulating and controlling, said relative positions for attaching said individual customized means for modulating and controlling to said individual customized means for implementing, said relative positions for rendering said spaces, said individual customized means comprising cornea-area-configured spaces between said upper and lower customized means for modulating and controlling, said spaces for avoiding exerting pressure on the corneas and lens so not to disrupt the physiology of said corneas and lens.

Said second networked repositories instructing said 3-D printers to render said spaces in said individual customized means for implementing integrated said means for modulating and controlling based on said analytics of said data, said spaces for avoiding exerting pressure on the corneas and lens so not to disrupt the physiology of said corneas and lens.

First example, a mid-decades-old Cantonese sagging-eyelids man living in Guangzhou, Guangdong Province, People's Republic of China, used his said networked smartphone for submitting his face selfies to New York City-situated first networked server repositories which instruct a Guangzhou-situated 3-D printer for manufacturing his one customized adjustable flexible hypoallergic elastic red-colored rubber domino mask-like band for fitting his idiosyncratic anatomical structures and contours of head, face, eyes, eyelids, nose, ears, eye orbits and surrounding bony and soft tissues as described herein. Instantly, said 3-D printer using rubber and red dye in additive manufacturing said red customized band contoured to have bilateral upper and lower crescent univalves between which are bilateral cornea-area-configured spaces for avoiding stressing the corneas and lenses, said spaces contoured to have a plurality of slits and holes for his sights, said bands contoured to loop bilateral ears as an anchoring means for optimizing the functions of said means for modulating and controlling and both free ends of said bands have various options of fasteners and clasps for adjustably uniting and disengaging said free ends and for varying said compression force being rendered by said apparatus for fitting to achieve the objectives and goals of the present invention. Any prescribed analgesics, anti-inflammatory drugs, antibiotics and any combination thereof can be impregnated or incorporated into said specific upper and/or lower parts and any combination thereof of said band during said printing or being printed as accessory of said band. Since said customized band for fitting said man is inexpensively printed, the requirement of removable and interchangeable parts is moot in this case.

Second example, an elderly Los Angeles woman who just had blepharoplasty used her networked smartphone for submitting her face selfies to Florida-situated first networked server repositories which instruct a Los Angeles-situated 3-D printer for manufacturing her one customized hypoallergic elastic blue-colored kinesiology tape for fitting her idiosyncratic anatomical structures and contours of her face, eyes, post-blepharoplasty eyelids, eye orbits and surrounding bony and soft tissues as described herein. Instantly, said 3-D printer using elastic fibers or tapes to print an one-piece customized skin-safe adhesive, hypoallergenic, latex-free tape for fitting said woman's idiosyncratic anatomical structures and contours. First preferred embodiment, said customized one-piece has an adhesive superior end portion for adjustably adhering to skin of the forehead and two adhesive inferior ends for adjustably adhering to the skin of the left face and the skin of the right face, said ends for varying said compression force being rendered by said apparatus for fitting, and bilateral customized bifurcated mid-portions being a bivalve (or being an upper univalve and a lower univalve) for fitting said idiosyncratic face and its anatomical parts between which are said cornea-area-configured spaces where no compression on the corneas and lens exists; second preferred embodiment, said customized one-piece has an adhesive superior end portion for adjustably adhering to skin of the forehead and two adhesive inferior ends for adjustably adhering to the skin of the left face and the skin of the right face, said ends for varying said compression force being rendered by said apparatus for fitting, and bilateral customized bifurcated mid-portions being attachment sites for bilateral bivalve (or bilateral upper and lower univalves) for fitting said idiosyncratic face and its anatomical parts between which are said cornea-area-configured spaces where no compression on the corneas and lens exists. For said second preferred embodiment, extra customized removable and interchangeable means for modulating and controlling—aka customized spare bivalves, univalves—can be manufactured at any time for this representative case by said 3-D printer and 3-D printers for her in Los Angeles and anywhere on earth.

Third example, yet another Thai man can have a plurality adhesive elastic tapes or individual adhesive elastic strips for attaching said means for modulating and controlling and for adhering to skin of said foreheads and faces, said tapes and strips have calibration for showing the length, said calibration for indicating specific positions along said tapes and strips for attaching said means for modulating and controlling. One of the preferred embodiments, first, each of said individual adhesive elastic tapes or individual adhesive elastic strips comprises at least two said left adhesive elastic tapes or strips and two right adhesive elastic tapes or strips, one end of each tape or strip for adjustably adhering to the skin of the forehead and the opposite end for adjustably adhering to the skin of contralateral cheek; second, the mid portions of said tapes or strips for holding said means for modulating and controlling to be anterior to bilateral eyes; third, each of said adhesive mid-portion is bifurcated into superior adhesive mid-portion tape or strip and inferior adhesive mid-portion tape or strip for attaching said means for modulating and controlling to be anterior to each eye and its eyelids. Said adjustable ends for varying said compression force being rendered by said apparatus for fitting on said eyelids. Extra customized removable and interchangeable means for modulating and controlling—aka customized spare bivalves, univalves—can be manufactured at any time for this representative case by said 3-D printer and 3-D printers for him in Bangkok and anywhere on earth.

Said tape/strip or tapes/strips henceforth collectively known as tapes comprise, but not limited to, kinesiology tapes, athletic tapes, sports tapes, surgical tapes, dressing tapes, and any commercially manufactured tapes, etc.

A plurality of receptacles for repeatedly receiving and housing said means for modulating and controlling being anterior to the eyes and eyelids is yet another preferred embodiment of said apparatus. Said receptacles are such as, but not limited to, holding sleeves, pockets, indentations, recesses and the likes for accepting and housing said means for modulating and controlling to achieve the goals and objectives of the invention.

Said woman and men—representing billions of people—can use said app for detecting and measuring compressive forces as described supra.

Said preferred embodiments in three examples represent other preferred embodiments described and not described herein. All preferred embodiments are for preventing and reducing sagging, droopy and puffy eyelids in people, post-blepharoplasty and post-surgeries of eyelids, for preventing and reducing visual changes and losses including presbyopia and for rejuvenating skin in said conditions in billions of people.

Said second networked repositories instructing said 3-D printers to impregnate individual customized means for modulating and controlling such as, but not limited to, bivalves, univalves, pads and patches with prescribed analgesics, non-steroidal and steroidal anti-inflammatory drugs or antibiotics and any combination thereof based on prescription data in said networked repositories associating with individual users of said apparatus. Said networked system of 3-D printers will use several commercial 3-D printing technologies such as, but not limited to, additive manufacturing. Said first networked repositories to create individual design data on individual customized means for modulating and controlling and means for implementing for fitting individual faces, eyes, eyelids, noses and ears, etc. of individuals. Said first networked repositories transmit said individual design data second networked repositories to instruct said 3-D printers to render additive manufacturing said individual customized apparatus for fitting. Various synthetic and man-made materials and components such as, but not limited to, various types of rubber, plastics and glasses, alloys, cotton and polyesters in various constitutions and combinations and states of matter are and will be commercially available for said 3-D printers to manufacture said apparatus. Networking to said networked repositories for serving billions of people, said omnipresent 3-D printers are located anywhere on earth where millions of customized apparatuses are rapidly and inexpensively produced by said ubiquitous 3-D printers in and adjacent to billions of users' domiciles or within said users' residences with more advanced personal computers anywhere on earth.

For example, first networked servers networking billions of smartphones, laptops and desktops comprising first networked repositories networking to said system of ubiquitous 3-D printers—networking or not networking to said second networked repositories networking to said first networked repositories—in millions of gas stations, supermarkets, stores, malls, towns, capitals, cities, states, provinces. etc. of all nations in all continents. Billions of individuals will have rapid and instant accesses to individual customized apparatuses being locally manufactured by said system of ubiquitous 3-D printers in and adjacent to their towns, cities, capitals, etc.

Although said networked system of ubiquitous 3-D printers is the most powerful preferred embodiments of present invention, it will be appreciated by and obvious to those skilled in the art and science that other types of commercial machines—with old and new technologies—in factories and machines in people's homes and will be and can be used also to manufacture said apparatus, parts and components to achieve the objectives of the invention without departing from the spirit and scope of the specification of the present invention.

The present invention enables any and all components described herein to be applicable, interchangeable and replaceable in and among devices to be manufactured and those devices commercially available in stores, retails and wholesale for decades. For example, spectacles and eyewears in people's possessions and readily available online and on the shelves, fix and rotating displays in retail stores such as, but not limited to, chains of pharmacy.

Although a few structures and shapes of the preferred embodiments of present invention were described, it will be appreciated by and obvious to those skilled in the art and science that adaptations, variations and derivatives of said embodiments are and will be made and manufactured to achieve the objectives of the invention without departing from the spirit and scope of the specification of the present invention.

Although one of the preferred embodiments of the present invention is the mobile devices for managing compressive forces being rendered to said organs and tissues, it will be appreciated by and obvious to those skilled in the art and science that other devices and machines for achieve supra will be manufactured and used to achieve the objectives of the invention without departing from the spirit and scope of the specification of the present invention.

Although one the preferred embodiments of present invention is described, it will be appreciated by and obvious to those skilled in the art and science that components, adaptations, variations and derivatives of said embodiments are and will be made and achieved to achieve the objectives of the invention without departing from the spirit and scope of the specification of the present invention.

Although one the preferred embodiments of present invention is described for people, it will be appreciated by and obvious to those skilled in the art and science that adaptations, variations and derivatives of said embodiments are and will be made and achieved for people and animals in similar or different settings, circumstances, anatomical parts and sites without departing from the spirit and scope of the specification of the present invention.

1. A system for preventing and reducing sagging, droopy and puffy eyelids and visual changes and losses and said conditions in pre- and post-blepharoplasty and pre- and post-surgeries of eyelids based on the physiology of and hemodynamics in and around the eyelids and eyes and surrounding soft tissues, said system comprising an apparatus comprising a plurality of different structures, said apparatus for modulating and controlling the circulations and volumes of blood and fluids in the eyes, eyelids and related soft tissues including connective tissues and adipose tissue, said apparatus for preventing and reducing the accumulations of fluids and growth, proliferation and expansion of said organs and soft tissues including connective tissues and adipose tissue in and around said organs and tissues, said apparatus for promoting, accelerating and attaining skin shrinkage in said conditions, said apparatus for preserving and restoring visual acuities and preventing and reducing visual losses, said apparatus for rendering said structures for avoiding the compression and disruptions of the physiology of the cornea and lens, a means for gauging modulation and control being generated by said apparatus on said organs and tissues, first networked repositories for receiving and analyzing data for predicting and indicating trends of said conditions and the needs of utilizations of said means by people based on said sciences and analyses rendered by said networked repositories, and a system of machines for rendering individual apparatuses for fitting individual idiosyncratic sizes, dimensions, contours and anatomical positions of said organs and tissues of individuals, first networked repositories networking to second networked repositories for instructing the repositories of said machines, said system comprises:
said means for modulating and controlling the circulations and volumes of blood and fluids in and preventing and reducing the accumulations of fluids and growth and expansion of said organs and soft tissues including connective tissues and adipose tissue in said organs and tissues to render and maintain certain shapes and curvatures of the eyes including lens and structures and dimensions of the skin of and said soft tissues in and adjacent to the eyes and eyelids, said means for modulating and controlling having a means for attaching to said means for implementing, wherein said means for modulating and controlling comprises configurations and surface areas for avoiding exerting pressure on the corneas and lens so not to disrupt the physiology of said corneas and lens;
a means for implementing said means for modulating and controlling to the diverse anatomical structures and contours of eyes, eyelids and surrounding bony and soft tissues wherein said means for implementing comprises configurations and surface areas for avoiding exerting pressure on the corneas and lens so not to disrupt the physiology of said corneas and lens by said means for modulating and controlling;
a means for attaching said means for modulating and controlling to said means for implementing;
said means for modulating and controlling is integrated into said means for implementing;
said means for modulating and controlling are adaptable to attach to and incorporate into ordinary headgears and eyewears owned by people;
a means for adjustably uniting and disengaging the free ends of said means for implementing to adaptably varying the compressions being rendered on said organs and tissues by said means for modulating and controlling;
said means for adjustably uniting and disengaging the free ends of said means comprising a means for utilizing the ears as an anchoring means for optimizing the functions of and positioning said means for modulating and controlling and said means for implementing;
a plurality of systems of receptacles for receiving and housing said means, said systems are located in specific positions for said systems for implementing said means for modulating and controlling;
a plurality of systems of calibrations for indicating and showing specific positions for attaching said means for modulating and controlling in and on said means for implementing;
various types of miniature means for measuring and informing in digital compressive forces being rendered to said organs and tissues by said means for modulating and controlling and said means for implementing;
a means for dispensing analgesics, anti-inflammatory drugs, antibiotics and any combination thereof to said eyes, eyelids and surrounding bony and soft tissues, said means for dispensing having a means for dispensing prescribed quantities of said analgesics, anti-inflammatory drugs or antibiotics and any combination thereof;
a means for dispensing anti-inflammatory modalities to said eyes, eyelids and surrounding bony and soft tissues, said means for dispensing having a means for dispensing prescribed quantities of said anti-inflammatory modalities;
networked servers, smartphones, laptops and desktops having first networked repositories having computer programs for receiving and analyzing data on genetics, phenotypes, lineages, facial and head images and videos including selfies and prescription data on said analgesics, anti-inflammatory drugs, antibiotics and any combination thereof, said first networked repositories having computer programs for predicting and extrapolating trends of said conditions in people, indicating and illustrating the needs of utilizations of said means in relevant people, computer programs for rendering said means to avoiding the compression and disruptions of the physiology of the corneas and lenses based on the analytics of said data;
said first networked repositories for disseminating said data including said trends and images and videos of said natural and disease-related conditions and losses and outcomes of said means for preventing and reducing;
networked servers comprising said second networked repositories networking to ubiquitous 3-D printers for rendering individual customized said apparatus for fitting individual faces, eyes and eyelids, said second networked repositories for instructing said 3-D printers to render said individual customized apparatus for fitting individual faces, eyes and eyelids based on instructions and specifications rendered in and by said first networked repositories, said second networked repositories for instructing said computer programs to rendering said means to avoiding the compression and disruptions of the physiology of the corneas and lenses in individuals based on said analytics of said data;
said apparatus for fitting and customized apparatus for fitting comprising customized means for modulating and controlling integrated with customized means for implementing,
said mobile devices app for causing said touch-sensitive thus pressure-sensitive screens to detect and measure compressive forces being rendered to said organs and tissues by said apparatus for fitting upon said screens contacting said means and said mobile devices app for displaying said measurements as digital data and voice data;
said mobile devices comprising electromagnetic wave-measuring devices, said devices to detect and measure the intensity of electromagnetic waves being emitted by said organs and tissues being compressed by said structures and to display measurements as digital data and voice data;
said mobile devices repositories for transmitting said data to said networked repositories; and
said networked repositories for managing said data in said networks.

2. The system according to claim 1 wherein said means for modulating and controlling comprises:
individual pairs of semi-elliptical, semi-circular or crescent semi-rigid or compressible adaptable bivalve or a pair of adaptable univalve eye pads or eye patches, each bivalve or said pair of univalve eye pad or patch comprising an upper adaptable, inflatable, soft or firm univalve eye pad or patch capable of attaching to and being reversibly removed from said means for implementing, each upper univalve eye pad or patch has configurations and surface areas approximating a plurality of surface areas of contacting upper eyelid, upper rim of the eye orbit, and varying widths of the palpebral fissure;
a lower adaptable, inflatable, soft or firm eye pad or patch capable of attaching to and being reversibly removed from said means for implementing, each lower univalve eye pad or patch has configurations and surface areas approximating a plurality of surface areas of contacting lower eyelid, lower rim of the eye orbit, and varying widths of the palpebral fissure;
each and every bivalve eye pads or patches is adaptable by having removable and interchangeable its univalve eye pads or patches to fit said diverse anatomical structures and contours of eyes, eyelids and surrounding bony and soft tissues;
said mobile device-touchscreen means for measuring and informing in digital compressive forces being rendered to said organs and tissues by said means for modulating and controlling and said means for implementing;
said means for modulating and controlling comprising said surface areas and a cornea-area-configured space between said upper and lower univalves for avoiding exerting pressure on the corneas and lens so not to disrupt the physiology of said corneas and lens;
said removable and interchangeable univalve eye pads or patches for avoiding exerting pressure on the corneas and lens so not to disrupt the physiology of said corneas and lens;
said means for implementing comprises configurations and surface areas for avoiding exerting pressure on the corneas and lens so not to disrupt the physiology of said corneas and lens by said means for modulating and controlling;
said means having a means for measuring and informing compressive forces being rendered by said means to said organs and tissues;
said means for attaching said means for modulating and controlling to said means for implementing;
a plurality of pads and patches containing said anti-inflammatory modalities;
said pads and patches for dispensing said anti-inflammatory modalities;
a plurality of pads and patches impregnated with said analgesics, anti-inflammatory drugs, antibiotics and any combination thereof; and
said pads and patches for dispensing said medications.

3. The system according to claim 1 wherein said means for modulating and controlling comprises:
individual pairs of semi-elliptical, semi-circular or crescent semi-rigid or compressible soft or firm synthetic bivalve inflatables or a pair of adaptable univalve inflatables, each bivalve inflatable or said pair of adaptable univalve inflatable comprising
an upper adaptable, inflatable, soft or firm synthetic univalve bladder capable of attaching to and being reversibly removed from said means for implementing, each upper univalve bladder has configurations and surface areas approximating a plurality of surface areas of contacting upper eyelid, upper rim of the eye orbit, and varying widths of the palpebral fissure;
a lower adaptable, inflatable, soft or firm synthetic univalve bladder capable of attaching to and being reversibly removed from said means for implementing, each lower univalve bladder has configurations and surface areas approximating a plurality of surface areas of contacting lower eyelid, lower rim of the eye orbit, and varying widths of the palpebral fissure;
each and every bivalve is adaptable by being removable and interchangeable to fit said diverse anatomical structures and contours of eyes, eyelids and surrounding bony and soft tissues;
each and every bivalve inflatable is adaptable by having removable and interchangeable its univalve inflatables to fit said diverse anatomical structures and contours of eyes, upper and lower eyelids and surrounding bony and soft tissues;
each and every univalve is adaptable by being removable and interchangeable to fit said diverse anatomical structures and contours of eyes, eyelids and surrounding bony and soft tissues;
said means for modulating and controlling comprising said surface areas and a cornea-area-configured space between said upper and lower univalves for avoiding exerting pressure on the corneas and lens so not to disrupt the physiology of said corneas and lens;
said removable and interchangeable bladder for avoiding exerting pressure on the corneas and lens so not to disrupt the physiology of said corneas and lens;
said means for implementing comprises configurations and surface areas for avoiding exerting pressure on the corneas and lens so not to disrupt the physiology of said corneas and lens by said means for modulating and controlling;
said means having various types means for measuring and informing compressive forces being rendered to said organs and tissues by said means for modulating and controlling and said means for implementing;
said means for attaching said means for modulating and controlling to said means for implementing;
said bladders for dispensing anti-inflammatory modalities by containing said modalities; and
said bladder for implementing said pads and patches for dispensing said analgesics, anti-inflammatory drugs, antibiotics and any combination thereof by being the reservoir and dispenser of said medications.

4. The system according to claim 1 wherein said customized apparatus for fitting comprises:
individual customized one-piece adhesive elastic tapes or adhesive elastic strips for fitting individual idiosyncratic sizes, dimensions and peculiar anatomical positions of said organs and tissues of individual users;
each one-piece has at least one adhesive superior end portion for adjustably adhering to skin of the forehead;
bilateral adhesive inferior ends for adjustably adhering to the skin of the left face and the skin of the right face, said adjustable adhesive ends for varying said compression force being rendered by said apparatus for fitting;
first embodiment, bilateral customized mid-portions of said tapes or strips comprise a plurality of receptacles for repeatedly receiving and housing said means for modulating and controlling being in anterior to both eyelids;
second embodiment, said bilateral customized bifurcated mid-portions of said tapes or strips being contoured into bilateral bivalve (or bilateral upper univalves and bilateral lower univalves) for fitting bilateral upper eyelids and lower eyelids between which are said cornea-area-configured spaces where no compression on the corneas and lens exists; and
third embodiment, said bilateral customized bifurcated mid-portions of said tapes or strips comprising bilateral upper and lower attachment sites for attaching a pair of bivalves (or bilateral upper univalves and bilateral lower univalves) for fitting bilateral upper eyelids and lower eyelids between which are said cornea-area-configured spaces where no compression on the corneas and lens exists.

5. The system according to claim 1 wherein said customized apparatus for fitting comprises:
a plurality adhesive elastic tapes for attaching said means for modulating and controlling, each adhesive tape has one adhesive end for adjustably adhering to the skin of the forehead and the opposite adhesive end for adjustably adhering to the skin of the face;
a plurality adhesive elastic strips for attaching said means for modulating and controlling, each adhesive strip has one adhesive end for adjustably adhering to the skin of the forehead and the opposite adhesive end for adjustably adhering to the skin of the face;
said tapes and strips have calibration for showing the length, said calibration for indicating specific positions along said tapes and strips for adjustably attaching said means for modulating and controlling;
at least two adhesive elastic tapes or adhesive strips whose adhesive ends for adjustably adhering to the skin of said forehead and the opposite ends for adjustably adhering to the skin of the left face;
at least two adhesive elastic tapes or adhesive strips whose adhesive ends for adjustably adhering to the skin of said forehead and the opposite ends for adjustably adhering to the skin of the right face;
the mid portions of said adhesive tapes or adhesive strips comprising receptacle means for receiving and housing a plurality of means for modulating and controlling to be anterior to bilateral eyes; or
the mid portions of said adhesive tapes or adhesive strips comprising a means for attaching said means for modulating and controlling to be anterior to bilateral eyes; or
each of adhesive mid-portions of said tapes or strips is bifurcated into superior adhesive mid-portion tape or strip and inferior adhesive mid-portion tape or strip, said bilateral bifurcated mid-portions for attaching said means for modulating and controlling to be anterior to the eyelids; and
said adhesive ends for varying said compression force being rendered by said apparatus for fitting on said eyelids.

6. The system according to claim 1 wherein said customized apparatus for fitting comprises:
individual pairs of commercially available spectacles applicable to people's faces and eyes, each pair of spectacles comprises a pair of frames and a pair of lenses;
an upper adaptable, soft synthetic petal-like semi-elliptical, semi-circular or crescent pad or patch capable of attaching to and being reversibly removed from said frame or said lens, the free-portion of said petal-like pad or patch has configurations and surface areas approximating a plurality of surface areas of contacting upper eyelid and varying widths of the palpebral fissure;
a lower adaptable, soft synthetic petal-like pad or patch capable of attaching to and being reversibly removed from said frame or said lens, the free-portion of said petal-like pad or patch has configurations and surface areas approximating a plurality of surface areas of contacting lower eyelid and varying widths of the palpebral fissure;
S each and every petal-like pad or patch petal-like semi-elliptical, semi-circular or crescent pad or patch is malleable to adapt to configurations and sizes of relevant eyelid, each and every petal-like pad or patch is adaptable by being removable and interchangeable with a plurality of petal-like pads or patches for fitting to diverse anatomical structures and contours of eyes, eyelids and surrounding bony and soft tissues;
said mobile device-touchscreen means for measuring and informing in digital compressive forces being rendered to said organs and tissues by said means for modulating and controlling and said means for implementing;
said means for modulating and controlling comprising said surface areas and a cornea-area-configured space between said upper and lower pads or patches for avoiding exerting pressure on the corneas and lens so not to disrupt the physiology of said corneas and lens;
adhesives for adjustably and reversibly adhering or attaching said petal-like pads or patches to said frames and lenses;
said frames and petal-like pads or patches comprise said configurations and surface areas for avoiding exerting pressure on the corneas and lens so not to disrupt the physiology of said corneas and lens;
said petal-like pad or patch comprise natural or man-made materials and any combination thereof;
adjustable flexible head bands and head straps for reversibly attaching to free ends of both long arms of said spectacles, said bands and straps having velcro fasteners or various configurations and types of plastic, metal and alloy clasps for adaptably varying the circumferences of a spectacles-band or a spectacles-strap for rendering pressures of said means for modulating and controlling on said eyes, eyelids and surrounding bony and soft tissues;
head bands and head straps comprising said means for utilizing the ears as an anchoring means for optimizing the functions of and positioning said means for modulating and controlling and said means for implementing;
said lenses may serve as receptacles for delivering said means for dispensing, having spaces into which a plurality of removable microwaveable synthetic cookies comprising cold or hot water, gel, chemical beads and air can be inserted into said spaces, said adjustable compressive forces and venous reductions are being rendered by adjustable numbers of said removable microwaveable cookies pads being inserted into said psi for indicating the desired adjustable compressive forces and venous reductions said bladder are moldable to the variable and diverse contours of the anatomical parts of the heads and faces;
a means for measuring said compressive forces and venous reductions being rendered by said means for compressing said arteries and reducing venous fluids and said adjustable flexible head bands or head straps having velcro fasteners or holes and buckles for adaptably varying said means for modulating and controlling, said velcro fasteners and holes having numeric and colored calibrations for correlating to digital displays of compression pressure in said digital means, said means having digital means for indicating and displaying said compressive forces being rendered by said means on said eyes, eyelids and surrounding soft tissues, said means for compressing having a means for dispensing anti-inflammatory modalities;
a plurality of pads and patches impregnated with said analgesics, anti-inflammatory drugs, antibiotics and any combination thereof;
said petal-like pads or patches for dispensing analgesics, anti-inflammatory drugs, antibiotics and any combination thereof; and
said means having various types of means for measuring and informing compressive forces being rendered to said organs and tissues by said means for modulating and controlling and said means for implementing.

7. The system according to claim 1 wherein said customized apparatus for fitting comprises:
individual goggle-like eyewears, each pair of eyewears comprises a pair of elliptical or circular frames applicable to each pair of eyes;
an upper adaptable, inflatable, soft or firm univalve eye pad or patch capable of attaching to and being reversibly removed from said means for implementing, each upper univalve eye pad or patch has configurations and surface areas approximating a plurality of surface areas of contacting upper eyelid, upper rim of the eye orbit, and varying widths of the palpebral fissure;
a lower adaptable, inflatable, soft or firm eye univalve pad or patch capable of attaching to and being reversibly removed from said means for implementing, each lower univalve eye pad or patch has configurations and surface areas approximating a plurality of surface areas of contacting lower eyelid, lower rim of the eye orbit, and varying widths of the palpebral fissure;
each and every bivalve eye pads or patches is adaptable by having removable and interchangeable its univalve eye pads or patches to fit said diverse anatomical structures and contours of eyes, eyelids and surrounding bony and soft tissues;
each and every bivalve is adaptable by being removable and interchangeable to fit said diverse anatomical structures and contours of eyes, eyelids and surrounding bony and soft tissues;
each and every univalve is adaptable by being removable and interchangeable to fit said diverse anatomical structures and contours of eyes, eyelids and surrounding bony and soft tissues;
said means for utilizing the ears as an anchoring means for optimizing the functions of and positioning said means for modulating and controlling and said means for implementing;
said mobile device-touchscreen means for measuring and informing in digital compressive forces being rendered to said organs and tissues by said means for modulating and controlling and said means for implementing;
said frames, pads and patches comprise said configurations and surface areas for avoiding exerting pressure on the corneas and lens so not to disrupt the physiology of said corneas and lens;
said pads and patches comprise natural or man-made materials and any combination thereof;
a pair of commercial clear transparent goggle lens may attach to and be reversibly removed from each pair said frames, said goggle lens may have ventilation means for equilibrating temperature and humidity to those of the ambience;
said means having various types of means for measuring and informing compressive forces being rendered to said organs and tissues by said means for modulating and controlling and said means for implementing;
said lens may serve as a receptacle for delivering said means for dispensing, having spaces into which a plurality of removable microwaveable cookies comprising cold or hot water, gel, chemical beads and air can be inserted into said spaces, said adjustable compressive forces and venous reductions are being rendered by adjustable numbers of said removable microwaveable cookies pads being inserted; and
said individual pairs of goggles comprising individual adjustable or interchangeable nosepieces for connecting said means for implementing said means for compressing said arteries and reducing venous fluids and an adjustable headpiece means for attaching said means to said eyes, eyelids and surrounding soft tissues, said headpiece means for adaptably adjusting amounts of said arterial compressions and venous fluid reductions.

8. The system according to claim 1 wherein said customized apparatus for fitting comprises:
individual goggle-like eyewears, each pair of eyewears comprises a pair of elliptical or circular frames applicable to each pair of eyes;
said upper adaptable, inflatable, soft or firm synthetic univalve bladder capable of attaching to and being reversibly removed from said means for implementing, each upper univalve inflatable has configurations and surface areas approximating a plurality of surface areas of contacting upper eyelid, upper rim of the eye orbit, and varying widths of the palpebral fissure;
said lower adaptable, inflatable, soft or firm synthetic univalve bladder capable of attaching to and being reversibly removed from said means for implementing, each lower univalve inflatable has configurations and surface areas approximating a plurality of surface areas of contacting lower eyelid, lower rim of the eye orbit, and varying widths of the palpebral fissure;
each and every bivalve inflatable is adaptable by having removable and interchangeable its univalve inflatable to fit said diverse anatomical structures and contours of eyes, eyelids and surrounding bony and soft tissues;
said means for utilizing the ears as an anchoring means for optimizing the functions of and positioning said means for modulating and controlling and said means for implementing;
said frames and inflatables comprise said configurations and surface areas for avoiding exerting pressure on the corneas and lens so not to disrupt the physiology of said corneas and lens;
said mobile device-touchscreen means for measuring and informing in digital compressive forces being rendered to said organs and tissues by said means for modulating and controlling and said means for implementing;
said inflatables comprise natural or man-made materials and any combination thereof;
a pair of commercial clear transparent goggle lens may attach to and be reversibly removed from each pair said frames, said goggle lens may have ventilation means for equilibrating temperature and humidity to those of the ambience;
said lens may serve as a receptacle for delivering said means for dispensing, having spaces into which a plurality of removable microwaveable cookies comprising cold or hot water, gel, chemical beads and air can be inserted into said spaces, said adjustable compressive forces and venous reductions are being rendered by adjustable numbers of said removable microwaveable cookies pads being inserted;
a means for measuring said compressive forces and venous reductions being rendered by said means for compressing said arteries and reducing venous fluids and said adjustable flexible head bands or head straps having velcro fasteners or holes and buckles for adaptably varying said means for modulating and controlling, said velcro fasteners and holes having numeric and colored calibrations for correlating to digital displays of compression pressure in said digital means, said means having digital means for indicating and displaying said compressive forces being rendered by said means on said eyes, eyelids and surrounding soft tissues, said means for compressing having a means for dispensing anti-inflammatory modalities;
said means having various types of means for measuring and informing compressive forces being rendered to said organs and tissues by said means for modulating and controlling and said means for implementing;
said bladders having said receptacle for dispensing anti-inflammatory modalities; and
said bladders having said receptacle for dispensing analgesics, anti-inflammatory drugs, antibiotics and any combination thereof.

9. The system according to claim 1 wherein said customized apparatus for fitting comprises:
adjustable flexible elastic domino mask-like bands or strips wherein the areas of the portion of said bands and strips anterior to bilateral eyes comprise a system of plurality of receptacles for accepting and housing upper and lower univalves in contact with bilateral eyes and bilateral upper and lower eyelids as described herein;
adjustable flexible elastic domino mask-like bands or strips wherein said areas of said bands and strips anterior to bilateral eyes comprises a plurality systems of calibrations for indicating and showing areas of said portion for reversibly attaching said univalves to contact bilateral eyes and bilateral upper and lower eyelids and surrounding bony and soft tissues as described herein;
individual pairs of semi-elliptical, semi-circular or crescent semi-rigid or compressible adaptable bivalve inflatables, pads, or patches and any combination thereof or a pair of adaptable univalve inflatables, eye pads or eye patches and any combination thereof, each bivalve or said pair of univalves comprising
an upper said univalve capable of attaching to and being reversibly removed from domino mask-like bands or strips, each upper univalve has configurations and surface areas approximating a plurality of surface areas of contacting upper eyelid, upper rim of the eye orbit, and varying widths of the palpebral fissure;
a lower said univalve capable of attaching to and being reversibly removed from domino mask-like bands or strips, each lower univalve has configurations and surface areas approximating a plurality of surface areas of contacting lower eyelid, lower rim of the eye orbit, and varying widths of the palpebral fissure;
each and every univalve is adaptable by being removable and interchangeable to fit said diverse anatomical structures and contours of eyes, eyelids and surrounding bony and soft tissues;
each and every bivalve is adaptable by being removable and interchangeable to fit said diverse anatomical structures and contours of eyes, eyelids and surrounding bony and soft tissues;
a plurality of systems of calibrations for indicating and showing specific positions for attaching said bladders, pads, or patches to said domino mask-like bands and strips, said bladders, pads, and patches having adhesive means for reversibly attaching to said bands and strips;
said configurations of said upper univalve and lower univalve, said configurations for avoiding exerting pressure on the corneas and lens so not to disrupt the physiology of said corneas and lens;
said positions of said upper univalve and lower univalve, said positions for avoiding exerting pressure on the corneas and lens so not to disrupt the physiology of said corneas and lens;
cornea-area-configured spaces between said upper univalve and lower univalve, said spaces for avoiding exerting pressure on the corneas and lens so not to disrupt the physiology of said corneas and lens;
said mobile device-touchscreen means for measuring and informing in digital compressive forces being rendered to said organs and tissues by said means for modulating and controlling and said means for implementing;
a plurality of slits and holes for vision between said upper univalve and said lower univalve may be implemented in said spaces in said domino mask-like bands and strips, said plurality of slits and holes for avoiding exerting pressure on the corneas and lens so not to disrupt the physiology of said corneas and lens;
said bands and strips comprise said means for utilizing the ears as an anchoring means for optimizing the functions of and positioning said means for modulating and controlling and said means for implementing;
the free ends of said bands and strips having various types of fasteners and clasps for adjustably uniting and disengaging said free ends, said unities for varying compressions being rendered on said organs and tissues by said masks, upper univalves and lower univalves;
a plurality of pads and patches containing said anti-inflammatory modalities;
said pads and patches for dispensing said anti-inflammatory modalities;
a plurality of pads and patches impregnated with said analgesics, anti-inflammatory drugs, antibiotics and any combination thereof; and said pads and patches for dispensing said medications.
said velcro fasteners and holes having numeric and colored calibrations for correlating to digital displays of compression pressure in said digital means, said means having digital means for indicating and displaying said compressive forces being rendered by said means on said eyes including lens, eyelids and surrounding bony and soft tissues, said means for compressing having a means for dispensing anti-inflammatory modalities;
adjustable flexible elastic domino-mask bands or strips having means for adjustably uniting and disengaging said bands for adaptably varying said means for modulating and controlling, said said velcro fasteners and holes having numeric and colored calibrations for correlating to digital displays of compression pressure in said digital means, said means having digital means for indicating and displaying said compressive forces being rendered by said means on said eyes including lens, eyelids and surrounding soft tissues, said means for compressing having a means for dispensing anti-inflammatory modalities;
adjustable flexible elastic head bands comprise a plurality of attachment sites for attaching said means for modulating and controlling to be anterior to bilateral eyes and their eyelids;
adjustable flexible elastic head bands comprise a plurality of receptacles for repeatedly receiving and housing said means for modulating and controlling along the lengths to be anterior to bilateral eyes and their eyelids;
said head bands have said configurations among a plurality of said configurations for attaching said means for modulating and controlling to avoid compression on the corneas and lens by said means for modulating and controlling;
said mobile device-touchscreen means for measuring and informing in digital compressive forces being rendered to said organs and tissues by said means for modulating and controlling and said means for implementing;
said head bands have a means for measuring and informing compressive forces being rendered to said organs and tissues by said means for modulating and controlling;
said head bands comprise a plurality of dimensions and sizes for fitting people's heads and faces;
said head bands comprises various configurations and types of commercially available and used hook-and-loop fasteners also known as Velcro, hook-and-pile fasteners and touch fasteners;
said head bands comprises various configurations and types of commercially available and used plastic, metal and alloy clasps;
said head bands comprises various configurations and types of commercially available and used holes and buckles;
said head bands comprises various configurations and types of commercially available and used adhesives;
said head bands comprises said configurations and surface areas for avoiding exerting pressure on the corneas and lens so not to disrupt the physiology of said corneas and lens;
said head bands comprise said configurations and surface areas for avoiding exerting pressure on the corneas and lens so not to disrupt the physiology of said corneas and lens by said means for modulating and controlling;
said head bands having a means for measuring and informing compressive forces being rendered by said means to said organs and tissues;
said head bands having said receptacles for dispensing anti-inflammatory modalities; and
said head bands having said receptacles for dispensing analgesics, anti-inflammatory drugs, antibiotics and any combination thereof; and
said means having various types of means for measuring and informing compressive forces being rendered to said organs and tissues by said means for modulating and controlling and said means for implementing.

10. The system according to claim 1 wherein said customized apparatus for fitting comprises:
individual adjustable flexible elastic upper hemi- or upper-half-domino mask-like bands or strips each of which comprising individual bilateral upper eyelid areas for attaching individual pairs of semi-elliptical, semi-circular or crescent semi-rigid or compressible adaptable upper univalve inflatables, pads, or patches and any combination thereof, said eyelid areas are united by individual adjustable nosepieces for anchoring said hemi-bands and hemi-strips means for optimizing the functions of and positioning upper univalve inflatables, pads, or patches and any combination thereof contacting said upper eyelids and surrounding bony and soft tissues and said means for implementing;
said plurality systems of calibrations for indicating and showing areas of said portion for reversibly attaching said upper univalves to contact bilateral upper eyelids and surrounding bony and soft tissues as described herein;
a plurality of receptacles for repeatedly receiving and housing said upper univalves;
said bands and strips comprise said means for utilizing the ears as an anchoring means for optimizing the functions of and positioning said upper univalves and said means for implementing;
the free ends of said bands and strips having various types of fasteners and clasps for adjustably uniting and disengaging said free ends, said unities for varying compressions being rendered on said organs and tissues by said masks, upper univalves and lower univalves;
a plurality of systems of calibrations for indicating and showing specific positions for attaching said univalves to said domino mask-like bands and strips, said bladders, pads, and patches having adhesive means for reversibly attaching to said bands and strips;
said upper hemi-bands and strips for avoiding exerting pressure on the corneas and lens so not to disrupt the physiology of said corneas and lens;
said configurations of said upper univalves, said configurations for avoiding exerting pressure on the corneas and lens so not to disrupt the physiology of said corneas and lens;
said positions of said upper univalves, said positions for avoiding exerting pressure on the corneas and lens so not to disrupt the physiology of said corneas and lens;
various types of miniature means for measuring and informing in digital compressive forces being rendered to said organs and tissues by said means for modulating and controlling and said means for implementing;
said mobile device-touchscreen means for measuring and informing in digital compressive forces being rendered to said organs and tissues by said means for modulating and controlling and said means for implementing;
a plurality of pads and patches containing said anti-inflammatory modalities;
said pads and patches for dispensing said anti-inflammatory modalities;
a plurality of pads and patches impregnated with said analgesics, anti-inflammatory drugs, antibiotics and any combination thereof; and
said pads and patches for dispensing said medications.

11. The system to claim 1 wherein said customized apparatus for fitting comprises:
individual adjustable flexible elastic lower hemi- or upper-half-domino mask-like bands or strips each of which comprising individual bilateral lower eyelid areas for attaching individual pairs of semi-elliptical, semi-circular or crescent semi-rigid or compressible adaptable lower univalve inflatables, pads, or patches and any combination thereof, said eyelid areas are united by individual adjustable nosepieces for anchoring said hemi-bands and hemi-strips means for optimizing the functions of and positioning lower univalve inflatables, pads, or patches and any combination thereof contacting said lower eyelids and surrounding bony and soft tissues and said means for implementing;
said plurality systems of calibrations for indicating and showing areas of said portion for reversibly attaching said lower univalves to contact bilateral upper eyelids and surrounding bony and soft tissues as described herein;
a plurality of receptacles for repeatedly receiving and housing said lower univalves;
said bands and strips comprise said means for utilizing the ears as an anchoring means for optimizing the functions of and positioning said upper univalves and said means for implementing;
the free ends of said bands and strips having various types of fasteners and clasps for adjustably uniting and disengaging said free ends, said unities for varying compressions being rendered on said organs and tissues by said masks, upper univalves and lower univalves;
a plurality of systems of calibrations for indicating and showing specific positions for attaching said univalves to said hemi-bands and strips, said univalves having adhesive means for reversibly attaching to said bands and strips;
said lower hemi-bands and strips for avoiding exerting pressure on the corneas and lens so not to disrupt the physiology of said corneas and lens;
said configurations of said lower univalves, said configurations for avoiding exerting pressure on the corneas and lens so not to disrupt the physiology of said corneas and lens;
said positions of said lower univalves, said positions for avoiding exerting pressure on the corneas and lens so not to disrupt the physiology of said corneas and lens;
said mobile device-touchscreen means for measuring and informing in digital compressive forces being rendered to said organs and tissues by said means for modulating and controlling and said means for implementing;
various types of miniature means for measuring and informing in digital compressive forces being rendered to said organs and tissues by said means for modulating and controlling and said means for implementing;
a plurality of pads and patches containing said anti-inflammatory modalities;
said pads and patches for dispensing said anti-inflammatory modalities;
a plurality of pads and patches impregnated with said analgesics, anti-inflammatory drugs, antibiotics and any combination thereof; and
said pads and patches for dispensing said medications.

12. The system according to claim 1 wherein said system of machines for rendering individual said apparatus for fitting individual idiosyncratic dimensions and peculiar anatomical positions of said organs and tissues of individuals comprises a system of 3-D printers for rendering said individual customized means for modulating and controlling and means for implementing, said individual customized apparatus for fitting said organs and tissues of people, said networked servers comprising said second networked repositories networking to ubiquitous 3-D printers for rendering said individual customized apparatus for fitting individual heads, faces, eyes, eyelids, noses, ears, computer programs attached to said second networked repositories, said computer programs for instructing said system of 3-D printers for rendering said individual customized means:
said second networked repositories for instructing said 3-D printers to render said individual customized apparatus for fitting based on instructions and specifications rendered in and by said first networked repositories;
said computer programs attached to said second networked repositories, said computer programs for instructing said 3-D printers for rendering said individual customized means for modulating and controlling and means for implementing for fitting said individual idiosyncratic dimensions and peculiar anatomical positions of said organs and tissues of individuals;
said computer programs for instructing said 3-D printers for rendering relative positions of said individual customized means for modulating and controlling, said relative positions for attaching said individual customized means for modulating and controlling to said individual customized means for implementing, said relative positions for rendering said spaces, said individual customized means comprising cornea-area-configured spaces between said upper and lower customized means for modulating and controlling; and
said computer programs for instructing said 3-D printers for rendering said spaces in said individual customized means for implementing integrated said means for modulating and controlling based on said analytics of said data, said spaces for avoiding exerting pressure on the corneas and lens so not to disrupt the physiology of said corneas and lens.

13. The system according to claim 1 wherein said networked devices comprising first networked repositories for receiving and analyzing data for predicting and indicating trends of said conditions and the needs of utilizations of said means by people comprises:
networked servers, smartphones, laptops and desktops having said first networked repositories comprising processor units and memories, computer programs attached to said processor units, said computer programs for instructing said processor units how to receive and analyze raw and processed data on images including selfies of faces of people based on lineages and patterns of said conditions and losses in various ethnicities, regions of the world, diseases and disorders, genotypes and phenotypes and ages;
said networked repositories for disseminating data including images and videos of said natural and disease-related conditions and visual changes and losses and outcomes of said means for preventing and reducing and preserving and restoring;
processor units for analyzing and sorting images of faces of said people based on data on the types and degrees of visual changes and images of eyelids referencing to said data;
said repositories having computer programs for instructing said processor units how to extrapolate and predict said occurrences referencing to various circumstances affecting individuals based on analytics of data;
said repositories having computer programs for instructing said processor units how to disseminate and share said data and results of said analytics including images and videos among networked servers, smartphones, laptops and desktops;
said memories for storing raw and processed data on images of faces of people based on lineages and patterns of said conditions and losses in various ethnicities, regions of the world, diseases and disorders, genotypes and phenotypes and ages; and
said mobile device-touchscreen means for measuring and informing in digital compressive forces being rendered to said organs and tissues by said means for modulating and controlling and said means for implementing, wherein individual mobile devices networked to said networked repositories transmitted data on individual compressive forces to said networked repositories.
networked servers, smartphones, laptops and desktops having first networked repositories having computer programs for receiving and analyzing data on genetics, phenotypes, lineages, facial and head images and video, said first networked repositories having computer programs for predicting and extrapolating trends of said conditions in people, indicating and illustrating the needs of utilizations of said means in relevant people, computer programs for rendering said means to avoiding the compression and disruptions of the physiology of the corneas and lenses based on the analytics of said data;
said first networked repositories for disseminating said data including said trends and images and videos of said natural and disease-related conditions and losses and outcomes of said means for preventing and reducing; and
networked servers comprising said second networked repositories networking to ubiquitous 3-D printers for rendering individual customized said apparatus for fitting individual heads, faces, eyes and eyelids, said second networked repositories for instructing said 3-D printers for rendering said individual customized apparatus for fitting individual faces, eyes and eyelids based on instructions and specifications rendered in and by said first networked repositories, said second networked repositories for instructing said computer programs to rendering said means to avoiding the compression and disruptions of the physiology of the corneas and lenses in individuals based on said analytics of said data.

14. The system according to claim 1 wherein said networked devices comprising first networked repositories for receiving and analyzing data for predicting and indicating trends of said conditions and the needs of utilizations of said means by people comprises:
said networked servers, smartphones, laptops and desktops having first networked repositories having computer programs for receiving and analyzing data on genetics, phenotypes, lineages, facial and head images and video and prescription data on said analgesics, anti-inflammatory drugs, antibiotics and any combination thereof, said first networked repositories having computer programs for predicting and extrapolating trends of said conditions in people, indicating and illustrating the needs of utilizations of said means in relevant people, computer programs for rendering said means to avoiding the compression and disruptions of the physiology of the corneas and lenses based on the analytics of said data; and
said networked servers comprising said second networked repositories networking to said 3-D printers for rendering individual customized said apparatus for fitting individual faces, eyes and eyelids, said second networked repositories for instructing said 3-D printers for impregnating said individual customized bivalves, univalves, pads and patches with said analgesics, anti-inflammatory drugs or antibiotics and any combination thereof based on prescription data in said networked repositories associating with individual users of said individual customized bivalves, univalves, pads and patches.

15. A system of networked repositories for receiving and analyzing data on demographics including genotypic and phenotypic data, facial images and videos including selfies in natural and disease-related conditions of individuals to predict and indicate trends of developments of eyebags and visual deficits and losses to indicate the needs of utilizations of an apparatus for preventing and reducing said conditions in individuals based on said analyses and for avoiding the compression of corneas and lenses whereby said networked repositories for networking to repositories of machines for producing individual customized apparatuses for fitting idiosyncratic dimensions, contours and relative positions of anatomical parts of the heads and faces of individuals, said system for providing devices for measuring and gauging compression forces being delivered by said apparatus on eyelids and eyes, said system comprises:
networked servers, smartphones, laptops and desktops comprising first networked repositories comprising processor units to which attached computer programs and memories, said first networked repositories for receiving and analyzing data on demographics, genetics, phenotypes, lineages of facial and head images and videos of individuals and prescription data on said analgesics, anti-inflammatory drugs, antibiotics and any combination thereof, said first networked repositories having computer programs for predicting and extrapolating trends of said conditions in people, indicating and illustrating the needs of utilizations of said means by relevant people, said computer programs rendering said structures of said means to avoid the compression and disruptions of the physiology of the corneas and lenses based on the analytics of said data;
networked servers comprising said second networked repositories networking to ubiquitous 3-D printers for rendering individual customized apparatus for fitting individual faces, eyes and eyelids, said second networked repositories for instructing said 3-D printers to render said individual customized apparatus for fitting individual faces, eyes and eyelids based on instructions and specifications rendered in and by said first networked repositories, said second networked repositories for instructing said computer programs to rendering said means to avoiding the compression and disruptions of the physiology of the corneas and lenses in individuals based on said analytics of said data;
said mobile devices app to adapt said touch-sensitive thus pressure-sensitive screens to detect and measure compressive forces being rendered to said organs and tissues by said structures upon said screens contacting said structures and to display measurements as digital data and voice data;
said mobile devices comprising electromagnetic wave-measuring devices, said devices to detect and measure the intensity of electromagnetic waves being emitted by said organs and tissues being compressed by said structures and to display measurements of said emissions
as digital data and voice data;
said mobile devices repositories for transmitting said data to said networked repositories;
said networked repositories for managing said data in said networks;
said means for modulating and controlling the circulations and volumes of blood and fluids in and preventing and reducing the accumulations of fluids and growth and expansion of said organs and soft tissues including connective tissues and adipose tissue in said organs and tissues to render and maintain certain shapes and curvatures of the eyes including lens and structures and dimensions of the skin of and said soft tissues in and adjacent to the eyes and eyelids, said means for modulating and controlling having a means for attaching to said means for implementing, wherein said means for modulating and controlling comprises configurations and surface areas for avoiding exerting pressure on the corneas and lens so not to disrupt the physiology of said corneas and lens;
said means for implementing said means for modulating and controlling to the diverse anatomical structures and contours of eyes, eyelids and surrounding bony and soft tissues wherein said means for implementing comprises configurations and surface areas for avoiding exerting pressure on the corneas and lens so not to disrupt the physiology of said corneas and lens by said means for modulating and controlling;
said means for attaching said means for modulating and controlling to said means for implementing;
said means for modulating and controlling is integrated into said means for implementing;
said apparatus for fitting and customized apparatus for fitting comprising customized means for modulating and controlling integrated with customized means for implementing.
said means for modulating and controlling adaptable to attach and be incorporated into ordinary headgears and eyewears owned by people;
a means for adjustably uniting and disengaging the free ends of said means for implementing to adaptably varying the compressions being rendered on said organs and tissues by said means for modulating and controlling;
a means for adjustably uniting and disengaging the free ends of said means comprising a means for utilizing the ears as an anchoring means for optimizing the functions of and positioning said means for modulating and controlling and said means for implementing;
a plurality of systems of receptacles for receiving and housing said means, said systems are located in specific positions for said systems for implementing said means for modulating and controlling;
a plurality of systems of calibrations for indicating and showing specific positions for attaching said means for modulating and controlling in and on said means for implementing;
various types of miniature means for measuring and informing in digital compressive forces being rendered to said organs and tissues by said means for modulating and controlling and said means for implementing;
a means for dispensing analgesics, anti-inflammatory drugs, antibiotics and any combination thereof to said eyes, eyelids and surrounding bony and soft tissues, said means for dispensing having a means for dispensing prescribed quantities of said analgesics, anti-inflammatory drugs or antibiotics and any combination thereof; and
a means for dispensing anti-inflammatory modalities to said eyes, eyelids and surrounding bony and soft tissues, said means for dispensing having a means for dispensing prescribed quantities of said anti-inflammatory modalities.

16. In a system for preventing and reducing sagging, droopy and puffy eyelids and visual changes and losses and said conditions in pre- and post-blepharoplasty and pre- and post-surgeries of eyelids, said system comprising an apparatus for preventing and reducing said conditions and rejuvenating eyelids based on the physiology of and hemodynamics in the eyelids and eyes and surrounding soft tissues, said apparatus having a plurality of structures for modulating and controlling the circulations and blood and fluids in said organs and tissues to prevent and reduce the accumulations of fluids in and growth, proliferation and expansion of soft tissues in and around said organs and tissues, a means for gauging modulation and control being generated by said structures, said apparatus for promoting, accelerating and attaining skin shrinkage in said conditions, said apparatus for preserving and restoring visual acuities and preventing and reducing presbyopia and visual losses, said apparatus comprising an avoidance means for not causing the compression and disruptions of the physiology of the corneas and lenses based on said sciences and analytics of data rendered in networked repositories, a method of said networked repositories receiving and analyzing data on demographics, facial images including selfies and videos in natural and disease-related conditions of individuals to predict and indicate trends of developments of said conditions to indicate the needs of utilizations of said apparatus in individuals whereby said networked repositories instructing a system of commercial machines including 3-D printers to produce individual customized apparatuses fitting idiosyncratic dimensions, contours and relative positions of anatomical parts of the heads and faces and avoiding said compression and disruptions based on said sciences and data, and measuring and gauging compression forces being delivered by said apparatus on the eyelids and eyes, said method comprises processes of:
attaching computer programs to processor units and memories of networked repositories of networked servers, smartphones, laptops and desktops, said computer programs instructing first networked repositories to receive and analyze data on demographics, genetics, phenotypes, lineages of facial and head images and videos of individuals and prescription data on said analgesics, anti-inflammatory drugs, antibiotics and any combination thereof, computer programs instructing networked repositories how to predict and extrapolate trends of said conditions in people based on said analyses and to indicate and illustrate the needs of utilizations of said system by individual users,
networking said networked servers the repositories for commanding and instructing ubiquitous commercial machines including 3-D printers to manufacture said idiosyncratic dimensions, contours and relative positions of anatomical parts of the heads and faces and avoiding said compression and disruptions for individual users based on instructions and specifications rendered in and by said first networked repositories and to render said apparatuses to avoid compressing and disrupting the physiology of the corneas and lenses in individuals based on said analyses of said data;
said computer programs instructing said 3-D printers to render said individual customized apparatuses fitting idiosyncratic dimensions, contours and relative positions to avoid the compression and disruptions of the physiology of the corneas and lenses based on said analyses of said data;
rendering an app in said mobile devices, said app adapting said touch-sensitive thus pressure-sensitive screens to detect and measure compressive forces being rendered to said organs and tissues by said structures upon said screens contacting said structures and to display measurements as digital data and voice data;
rendering electromagnetic wave-measuring devices in said mobile devices, said devices detecting and measuring the intensity of electromagnetic waves being emitted by said organs and tissues being compressed by said structures and to display measurements of said emissions as digital data and voice data;
said mobile devices repositories transmitting said data to said networked repositories;
said networked repositories managing said data in said networks;
rendering said individual customized apparatuses to comprise customized means for modulating and controlling integrated with customized means for implementing;
said apparatus utilizing said ears as an anchoring means to optimize the functions of and positions said apparatus;
rendering free ends of said apparatus to adaptably vary said compressions being rendered on said organs and tissues by said apparatus;
a plurality of systems of receptacles receiving and housing said means, said systems are located in specific positions for said systems for implementing said means for modulating and controlling;
a plurality of systems of calibrations indicating and showing specific positions for attaching said means for modulating and controlling in and on said means for implementing;
various types of miniature means measuring and informing compressive forces being rendered to said organs and tissues by said means for modulating and controlling and said means for implementing;
said apparatus dispensing analgesics, anti-inflammatory drugs, antibiotics and any combination thereof to said eyes, eyelids and surrounding bony and soft tissues,
said means dispensing prescribed quantities of said analgesics, anti-inflammatory drugs or antibiotics and any combination thereof; and
said means dispensing anti-inflammatory modalities to said eyes, eyelids and surrounding bony and soft tissues, said means for dispensing having a means dispensing prescribed quantities of said anti-inflammatory modalities;
rendering bivalves and univalves to be mobile and interchangeable to fit said diverse anatomical dimensions, sizes, structures and contours of heads, faces, eyes, eyelids and surrounding bony and soft tissues;
rendering said bivalves to attach and be incorporated into ordinary headgears and eyewears owned by people; and rendering said univalves to attach and be incorporated into ordinary headgears and eyewears owned by people.
